(19) United States
(12) Patent Application Publication
(10) Pub. No.: US 2017/0165100 A1
(43) Pub. Date: Jun. 15, 2017

(54) Urinary Absorbent and Retention Pouch for Male Incontinence

(71) Applicants: Cathy Jackson, Richardson, TX (US); John Wilkinson, Louisville, KY (US)

(72) Inventors: Cathy Jackson, Richardson, TX (US); John Wilkinson, Louisville, KY (US)

(21) Appl. No.: 15/376,117

(22) Filed: Dec. 12, 2016

Related U.S. Application Data
(60) Provisional application No. 62/265,482, filed on Dec. 10, 2015.

(51) Int. Cl. A61F 5/453; A61F 5/44

(52) U.S. Cl. CPC A61F 5/453; A61F 5/4401; A61F 5/4408

(57) ABSTRACT

A urinary absorbent and retention pouch for male incontinence is provided. The urinary absorbent and retention pouch includes an expandable pocket for containing male genitalia and absorbing and retaining urine discharge resulting from male incontinence. The pocket includes a flexible and elastic material for enabling the pocket to stretch. An elastic band defines an opening for providing access into the pocket and provides sufficient tension to sustain the pouch around the male genitalia. The pocket includes an absorbent interior layer for absorbing secreted urine and a liquid-impermeable exterior layer for retaining the urine in the pouch and preventing soiling. A removably attachable flap provides access to the male genitalia while the pouch is worn. A plurality of accordion pleats enables the pocket to expand its shape while in motion. A fastener enables the pocket to be suspended from a waistband for added support.

### CROSS REFERENCE TO RELATED APPLICATIONS

This application claims the benefit of U.S. Provisional Application No. 62/265,482 filed on Dec. 10, 2015. The above identified patent application is herein incorporated by reference in its entirety to provide continuity of disclosure.

### BACKGROUND OF THE INVENTION

The present invention relates to male urinary absorbent devices. More specifically, the present invention relates to a male urinary pouch for absorbing and retaining urine discharge resulting from male urinary incontinence.

Male urinary incontinence may be caused by a variety of factors. It can occur, in varying degrees, in infant males not yet toilet trained, young boys suffering from nocturnal enuresis or bed wetting, men who have undergone a prostatectomy or other urinary tract surgeries, such as a cystectomy, in diabetics with nerve damage, and in men who have had a stroke or spinal cord injury or who suffer from Parkinson's disease or multiple sclerosis. Aging is also a leading cause of male incontinence. Other causes include loss of control from such actions as coughing, sneezing, and lifting, and an abnormal and overwhelming urge to urinate caused, for example, by various bladder conditions such as exposure to radiation therapy. Finally, men who are handicapped or otherwise medically compromised may have problems with incontinence. Urinary incontinence can be more than an inconvenience and an embarrassment to men, it can cause discomfort from being in wet or damp clothing as well as genital itching or a rash or infection caused by contact with discharged urine.

Conventional absorbent devices to help manage male urinary incontinence include those which are positioned beneath undergarments and next to or over the genitals. Discharged urine is retained within an absorbent layer while a moisture barrier layer is interposed between the retained urine and the undergarments and other clothing. In this way, discharged urine is retained and not allowed to leak onto, or otherwise soil, clothing. In addition, the anxiety and potential embarrassment of being seen with soiled clothing is avoided. The used absorbent device is then later disposed of at an appropriate time. Such devices include diapers, which enclose the genitals, perineum, and buttocks, and pads, which enclose only the genital region.

While such absorbent devices may be effective in absorbing urine and preventing soiled clothing, a bulky or protruding appearance may call attention to the problem, the genitals may be allowed to come into contact with discharged urine, with resultant discomfort and the chance of rash or infection, and the device may cause further discomfort by not remaining in place while allowing the wearer to move about freely and engage in normal daily activities. Further, these absorbent devices are not stretchable, causing discomfort for individuals having bigger male genitalia or for those who frequently experience erections.

Thus, there is a need for an improved male urinary incontinence device that is not only effective, but that fits the male genital anatomy comfortably, allows sufficient freedom of movement to permit normal daily activities, may be worn discretely, is expandable and stretchable for accommodating larger male genitalia and erections, and that prevents the genitals from coming into contact with discharged urine.

### SUMMARY OF THE INVENTION

In view of the foregoing disadvantages inherent in the known types of male urinary absorbent device now present in the prior art, the present invention provides a urinary absorbent and retention pouch wherein the same can be utilized for providing convenience for the user when experiencing male incontinence. The present invention comprises a urinary absorbent and retention pouch, including, but not limited to, a pocket that defines an interior volume sized to receive and retain male genitalia therein. The pocket includes a proximal end, a distal end, an absorbent layer, and a liquid impermeable layer. The absorbent layer is disposed on an interior of the pocket and is configured to absorb urine discharged from male genitalia. The liquid-impermeable layer is disposed on an exterior of the pocket and is configured to retain the urine discharge within the pocket. The pocket is composed of a flexible material having elasticity for enabling the pocket to stretch and expand. A first opening is disposed at the proximal end of the pocket, which provides access into the interior volume of the pocket. The first opening is defined by an elastic band. A plurality of accordion pleats are disposed on the pocket and enable the pocket to expand perpendicularly relative to the accordion pleats. A removably attachable flap is hingedly connected to a top end of the pocket and provides access to a second opening disposed on the top end of the pocket. The second opening provides access to the interior volume of the pocket.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a front perspective view of the urinary absorbent and retention pouch.

FIG. 2 shows show a rear perspective view of the urinary absorbent and retention pouch.

FIG. 3 shows a phantom and cross-sectional view of the pocket of the urinary absorbent and retention pouch.

FIG. 4 shows a perspective view of the flap of the urinary absorbent and retention pouch as unattached to the pocket.

FIG. 5A shows a perspective view of the pouch as fastened to a waistband.

FIG. 5B shows a perspective view of an alternative embodiment of the pouch including a waistband.

FIG. 6 shows a perspective view of an alternative embodiment of the pouch including a drawstring.

FIG. 7 shows a perspective view of an alternative embodiment of the pouch including widthwise accordion pleats.

### DETAILED DESCRIPTION OF THE INVENTION

Reference is made herein to the attached drawings. Like reference numerals are used throughout the drawings to depict like or similar elements of the urinary absorbent and retention pouch. The figures are intended for representative purposes only and should not be considered to be limiting in any respect.

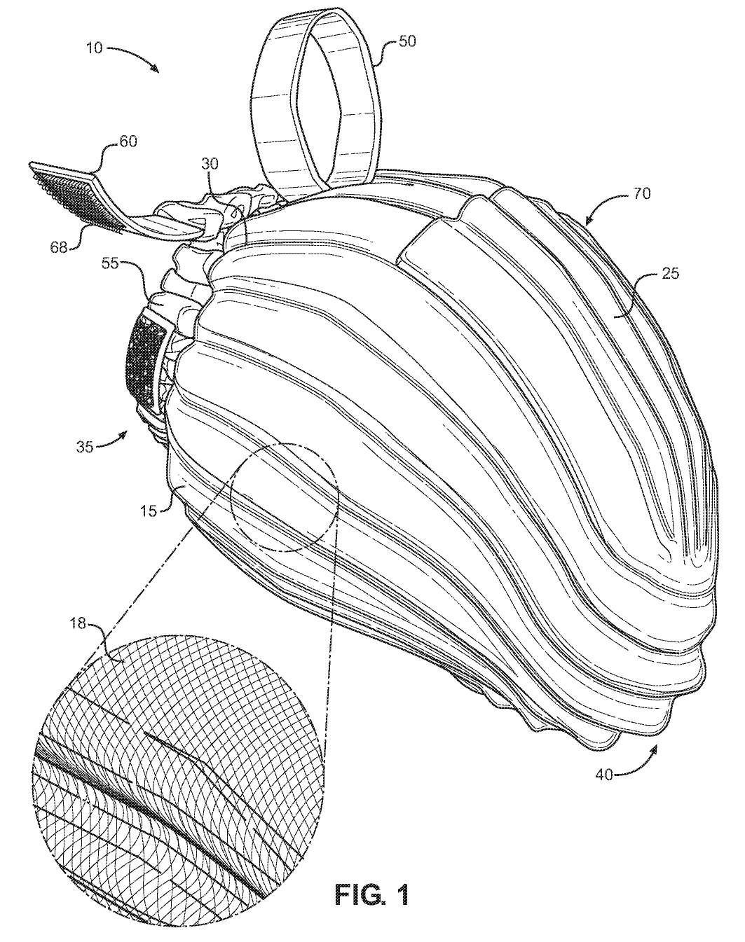

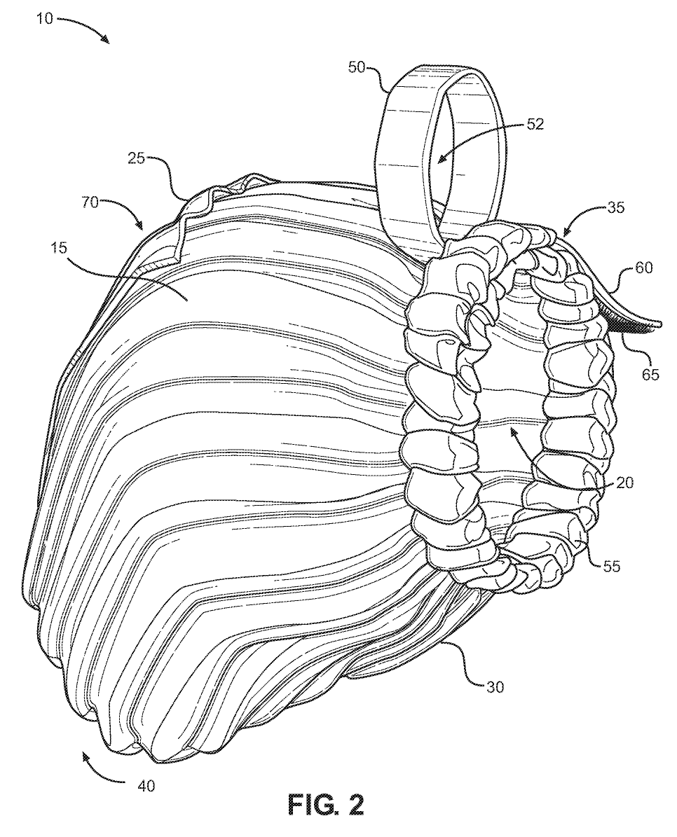

Referring now to FIGS. 1-2, there is shown a front perspective view and a rear perspective view, respectively, of the urinary absorbent and retention pouch. The urinary absorbent and retention pouch 10 includes an expandable pocket 15 for containing male genitalia and absorbing and retaining urine discharge resulting from male incontinence, an opening 20 for providing access into the pocket 15 and donning the pouch 10, a flap 25 for providing access to the male genitalia while the pouch 10 is worn, and a plurality of accordion pleats 30 that enables the pocket 15 to expand its shape.

The pocket 15 includes and interior volume sized to receive and retain the male genitalia therein. The pocket 15 is composed of a flexible unitary piece of material 18 such that the pocket falls over, or takes the shape of, the male genitalia when donned thereover, and does not form a bulge therearound. The pocket 15 is further composed of an elastic material 18 that enables the pocket 15 to stretch with the male genitalia in the event of an erection.

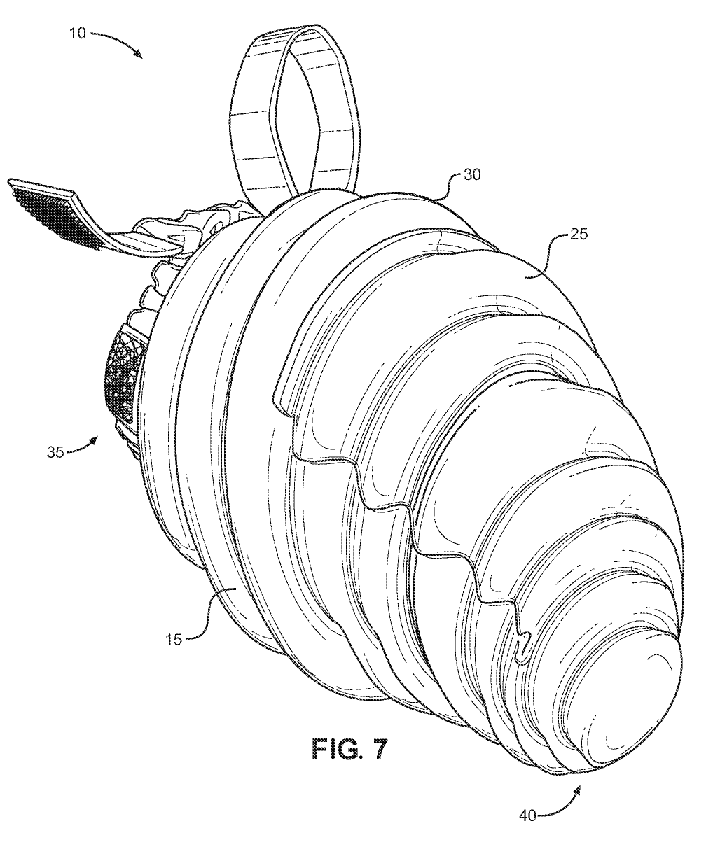

In the depicted embodiment, the plurality of accordion pleats 30 extend along the longitudinal length of the pocket 15 from a proximal end 35 of the pocket 15 to a distal end 40 of the pocket 15, forming an accordion like fold (or fan-like fold) along the longitudinal length of the pocket 15. In operation, the accordion pleats 30 enable the pocket 15 to expand perpendicularly relative to the accordion pleats 30 while in motion, such that the distal end 40 expands upwards and away from the proximal end 35 or away from the user's body when in use. In this way, the pouch 10 provides a means by which the pocket 15 can expand to accommodate the movement of the male genitalia while a user is in motion and facilitate the stretching of the pocket 15 when a user experiences an erection. In another embodiment, the plurality of accordion pleats 30 extend widthwise, or perpendicular to the longitudinal axis of the pocket 15, from the proximal end 35 to the distal end 40, as shown by FIG. 7. The accordion pleats 30 form an accordion-like fold along the width of the pocket 15, which enables the pocket 15 to expand longitudinally while in motion, such that the distal end 40 expands outwardly and away from the proximal end 35, or away from the user's body, when in use.

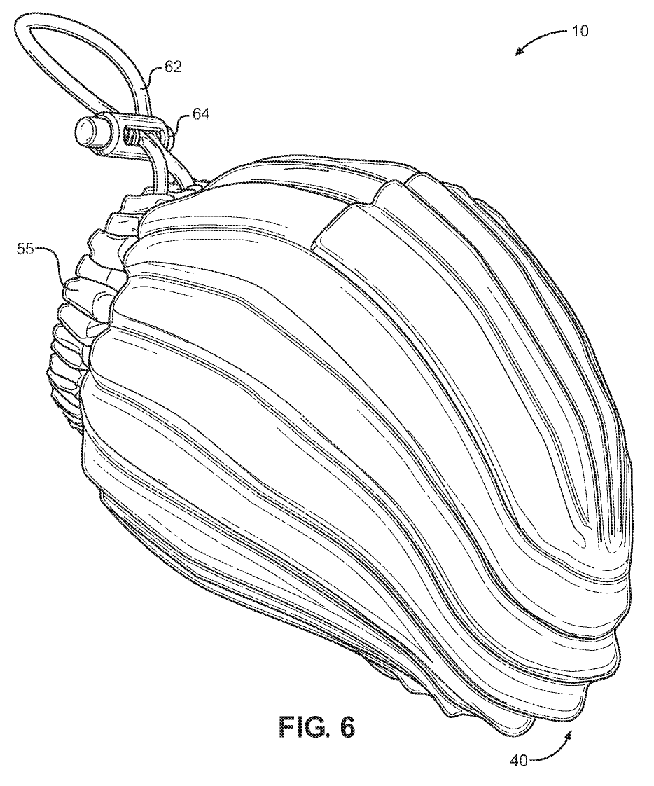

The opening 20 is defined by an elastic band 55. The elastic band 55 includes sufficient tension to sustain the pouch 10 around the male genitalia, such that the male genitalia does not slide out from the opening 20, but not so tight as to create discomfort. In one embodiment, the elastic band 55 includes an adjustment strap 60 having a fastener for adjusting the diameter of the opening 20 and further tightening the elastic band 55 around the male genitalia. In the depicted embodiment, the adjustment strap 60 includes a male hook and loop fastener 65 and the elastic band 55 includes a complimentary female hook and loop fastener 68 therealong for receiving the male hook and loop fastener at a desired length and adjusting the tightness of the elastic band 55 around the male genitalia. In another embodiment, the elastic band 55 includes a drawstring 62 having an adjustable clasp 64 for adjusting the drawstring, as shown in FIG. 6, which enables a user to adjust the diameter of the opening around the male genitalia.

The pocket 15 further includes a fastener for suspending the pocket 15 from a waistband and providing vertical support to the pouch 10 when in use. In the depicted embodiment, the fastener includes a loop 50 disposed on a top end 70 of the pocket 15 between the flap 25 and the opening 20. In the depicted embodiment, the loop 70 abuts the elastic band 55. The loop 50 includes a raised piece of material protruding radially outwardly relative to the pocket 15 that forms an aperture 52 configured to receive a waistband therethrough. In one embodiment, the loop 50 is sewn to the pocket 15. In another embodiment, the loop 50 is integral to, or a part of, the pocket 15. In alternative embodiments, the fastener includes a hook and loop fastener, such as Velcro, configured to receive a complimentary, mating hook and loop fastener.

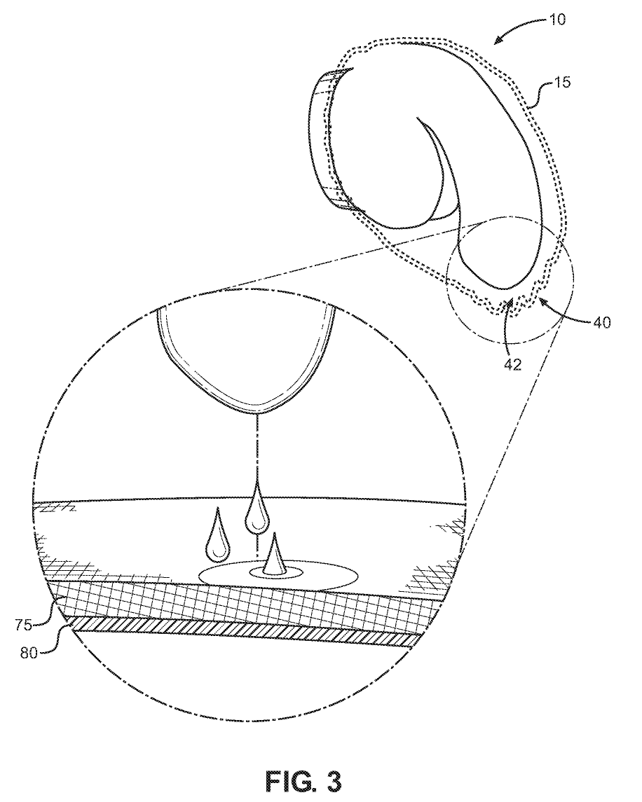

Referring now to FIG. 3, there is shown a phantom and cross-sectional view of the pocket. The pocket 15 includes an absorbent layer 75 on an interior thereof and a liquid-impermeable layer 80 on an exterior thereof. The absorbent layer 75 absorbs any urine discharged from the penis, while the liquid-impermeable layer 80 prevents the liquid absorbed by the absorbent later 75 from seeping out of the pouch 10 and soiling a user's clothes. In one embodiment, the distal end 40 of the pocket 15 includes a cavity 42 for collecting discharged urine therein. The cavity 42 includes a smaller diameter than the interior volume of the pocket 15 and is configured to rest below the male genitalia such that it prevents any urine absorbed by the pocket 15 from making contact with the male genitalia.

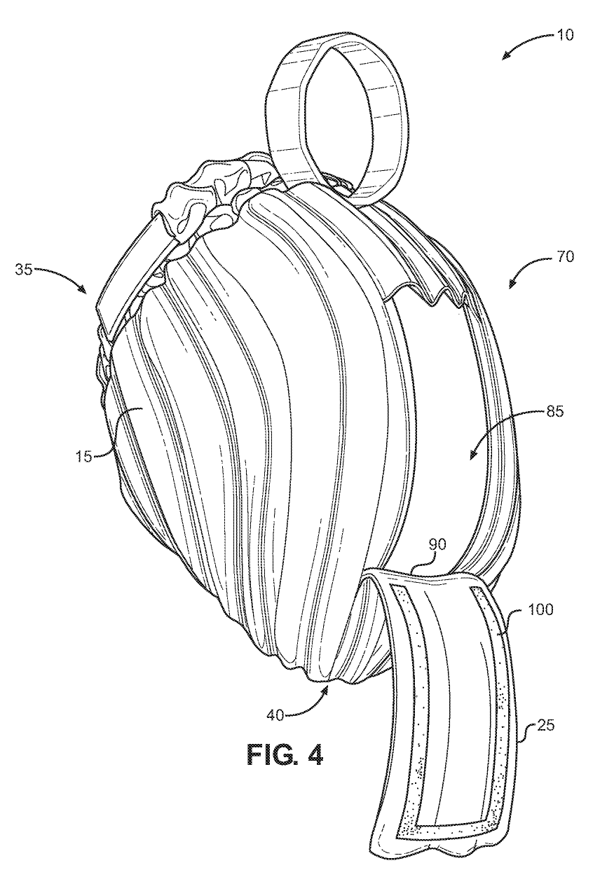

Referring now to FIG. 4, there is shown a perspective view of the flap as unattached to the pocket. The flap 25 is disposed on the top end 70 of the pocket 15 and encloses an opening 85. The flap 25 is hingedly connected to the top end 70 of the pocket 15 and includes a fastener 100 for removably attaching the flap 25 to the pocket 15. In the depicted embodiment, the flap 25 is hingedly connected via a living hinge 90 and positioned widthwise relative to the pocket 15 and adjacent to the distal end 40, such that when the pouch 10 is donned, the flap 25 opens radially outwardly towards the distal end 40 of pocket 15. In a second embodiment, the living hinge 90 is be positioned lengthwise relative to the pocket 15 such that the flap 25 opens perpendicularly relative to longitudinal axis of the pocket 15. In alternative embodiments, the living hinge 90 is positioned widthwise and adjacent to the proximal end 35, such that when the pouch 10 is donned, the flap 25 opens radially outwardly towards the proximal end 35 of the pocket 15. When attached to the pocket 15, the flap 25 encloses the opening 85 and prevents the unwanted egression of male genitalia therefrom while the pouch 10 is in use. The flap 25 comprises the same flexible and elastic material and characteristics as the pocket 15 so as to expand, stretch, and move with the pocket 15 while in motion or in use.

The opening 85 is disposed on the top end 70 of the pocket 15 and positioned between the proximal end 35 and distal end 40. The opening 85 provides access to interior volume of the pocket 15, such that a user may access and remove his genitalia from the pocket 15 while the pouch 10 is donned. The fastener 100 enables a user to open and close the opening 85 as desired via the flap 25. In one embodiment, the fastener 100 comprises a water-resistant or hydrophobic adhesive material configured to prevent any urine from seeping out of the pouch 10 through the flap 25. In another embodiment, the fastener 100 is disposed along the entire edge of the flap 25, such that the flap 25 may completely enclose the opening 85 and prevent any leakage therefrom. In the depicted embodiment, the flap 25 includes a rectangular flap having an adhesive material disposed around the entire periphery edge thereof for adhering to the pocket 15 and completely enclosing the opening 85.

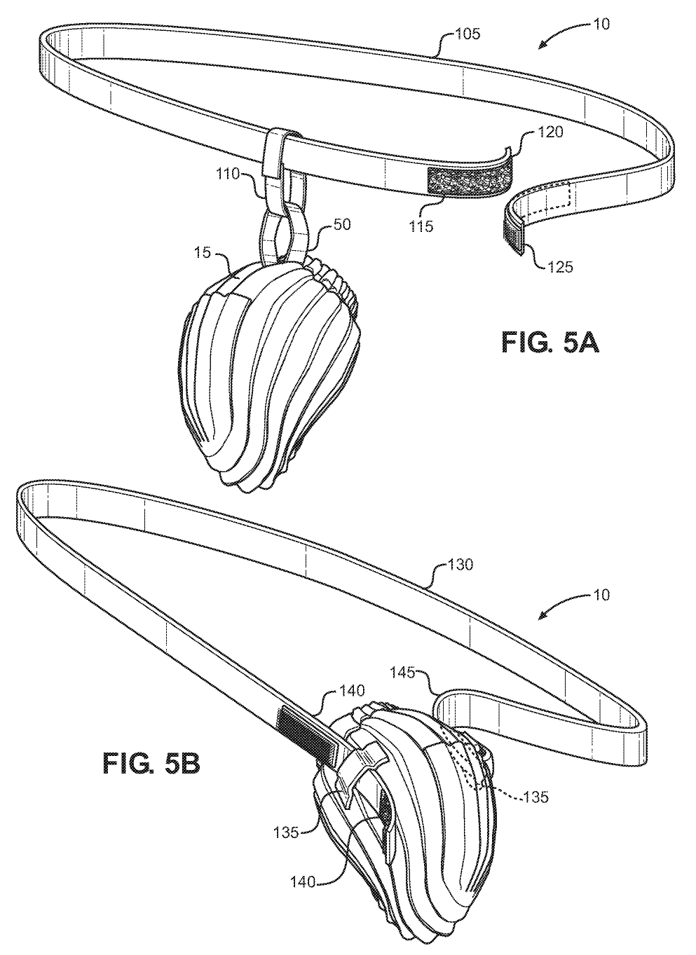

Referring now to FIG. 5A, there is shown a perspective view of the pouch as fastened to a waistband. In one embodiment of the present invention, the pouch 10 includes a waistband 105 configured to secure around a user's waist and a strap 110 for attaching the waistband 105 to the pouch 10 and suspending the pocket 15 therefrom. The waistband 105 provides vertical support to the pouch 10 when donned over male genitalia. In this way, the waistband 105 may provide added support to a pouch 10 retaining a lot of urine. The waistband 105 includes a fastener 115 for securing the waistband 105 around a user's waist. In the depicted embodiment, the waistband 105 comprises a flat piece of material including a first end 120 and a second end 125 having a complimentary, mating hook and loop fasteners disposed thereon for securing the ends 120, 125 to one another.

The strap 110 includes a thin piece of material having a length less than the length of the waistband 105. The strap 110 includes a first end and a second end having complimentary, mating hook and loop fasteners for the securing the ends to one another. In order to suspend the pouch 10 from the waistband 105 to provide the pouch 10 support, the waistband is first fastened around the waist. The strap 110 is then threaded through the loop 50 of the pocket 15. Once threaded through the loop 50, the strap 110 may be fastened around the waistband 105, thereby suspending the pouch 10 from the waistband 105. In this way, the strap 110 provides an extension in between the waistband 105 and the loop 50, such that it provides the pocket 15 slack when utilizing the waistband 105 to suspend the pocket 15 therefrom.

Referring now to FIG. 5B, there is shown a perspective view of an alternative embodiment of the pouch. In an alternative embodiment of the present invention, the pouch 10 includes waistband 130 and a pair of loops 135 positioned on opposing sides or bilaterally on the pocket 15 that are configured to receive the waistband 130 so as to provide further vertical support to the pouch 10. The loops 135 include a raised piece of material forming an aperture extending perpendicularly relative to the longitudinal axis of the pocket 15, or widthwise. The waistband includes a first end 140 and a second end 145, each including a male and female hook and loop fastener for folding the end onto itself and securing it thereto. The loops 135 are configured to receive either ends 140, 145 of the waistband 130 therethrough, such that the each end may fold around a loop 135 and fasten to itself, thereby securing the waistband 130 to the pouch 10. In operation, the pouch 10 is first donned and the waistband 130 is placed around the waist. Then, the ends 140, 145 of the waistband 130 are threaded through the apertures of the loops 135 and folded around the loop and back onto themselves to fasten the ends 140, 145 around the loops 135.

It is therefore submitted that the instant invention has been shown and described in various embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A urinary absorbent and retention pouch, comprising:
a pocket defining an interior volume sized to receive and retain male genitalia, the pocket including a proximal end, a distal end, an absorbent layer, and a liquid impermeable layer;
the absorbent layer disposed on an interior of the pocket, the absorbent layer configured to absorb urine discharged from the male genitalia;
the liquid-impermeable layer disposed on an exterior of the pocket, the liquid-impermeable layer configured to retain the urine discharge within the pocket;
wherein the pocket is composed of a flexible and elastic material for enabling the pocket to stretch and expand;
a first opening disposed at the proximal end of the pocket for providing access into the interior volume of the pocket, the first opening defined by an elastic band;
a plurality of accordion pleats disposed on the pocket, the accordion pleats enabling the pocket to expand perpendicularly relative thereto; and
a removably attachable flap hingedly connected to a top end of the pocket, the flap providing access to a second opening disposed on the top end of the pocket, the second opening providing access to the interior volume of the pocket.

2) The urinary absorbent and retention pouch of claim 1, wherein the distal end forms a cavity on an interior of the pocket, the cavity including a smaller diameter than the diameter of the interior volume of the pocket and configured to rest below the male genitalia and receive urine discharge therein, such that the cavity prevents the urine from contacting the male genitalia.

3) The urinary absorbent and retention pouch of claim 1, wherein the elastic band comprises an adjustment strap including a fastener for adjusting a diameter of the first opening.

4) The urinary absorbent and retention pouch of claim 1, wherein the elastic band comprises a drawstring including an adjustment clasp for adjusting a diameter of the first opening.

5) The urinary absorbent and retention pouch of claim 1, wherein the accordion pleats extend along a longitudinal length of the pocket from the proximal end to the distal end.

6) The urinary absorbent and retention pouch of claim 1, wherein the accordion pleats extend widthwise along the pocket.

7) The urinary absorbent and retention pouch of claim 1, further comprising a fastener disposed on a center axis of a top end of the pocket, the fastener configured to secure a waistband to the pocket and suspend the pocket therefrom.

8) The urinary absorbent and retention pouch of claim 7, wherein the fastener includes a raised piece of material extending along a longitudinal axis of the pocket, the raised piece of material defining an aperture configured to receive a waistband therethrough.

9) The urinary absorbent and retention pouch of claim 8, further comprising:
a waistband for securing around a user's waist;
a strap for threading through the aperture formed by the raised piece of material and fastening to the waistband;
wherein the waistband and strap provide a vertical support structure for the pocket.

10) The urinary absorbent and retention pouch of claim 1, wherein the flap includes a fastener disposed on an edge thereof, the fastener configured to enclose the flap around the second opening.

11) The urinary absorbent and retention pouch of claim 10, wherein the fastener comprises an adhesive material.

12) The urinary absorbent and retention pouch of claim 1, wherein the flap is composed of the same material as the pocket.

13) The urinary absorbent and retention pouch of claim 1, further comprising:
a waistband including a first end and a second end, the first end including a mating fastener and the second end including a complimentary mating fastener;
a pair of raised piece of material positioned on opposing lateral sides of the pocket, the pair of raised piece of material each defining an aperture;
wherein the first and second ends are configured to be threaded through the apertures and fastened around the raised piece of materials so as to suspend the pocket from the waistband.
